(19) United States
(12) Patent Application Publication
(10) Pub. No.: US 2024/0148003 A1
(43) Pub. Date: May 9, 2024

(54) ENDOPHYTIC BACTERIAL STRAINS, PROBIOTIC MIXTURES, FORMULATION AND METHOD, FOR STIMULATING PLANT GROWTH

(71) Applicant: Cristóbal FONSECA SEPÚLVEDA, Jalisco (MX)

(72) Inventors: Cristóbal FONSECA SEPÚLVEDA, Zapopan (MX); Gloria Margarita MACEDO RAYGOZA, Atotonilco el Alto (MX)

(21) Appl. No.: 18/281,464

(22) PCT Filed: Mar. 12, 2021
(86) PCT No.: PCT/MX2021/000011

§ 371 Date: Sep. 11, 2023

(30) Foreign Application Priority Data
Mar. 11, 2021 (MX) MX/A/2021/002958

(51) Int. Cl. A01N 63/20; C12N 1/20; C12R 1/01

(52) U.S. Cl. CPC A01N 63/20; C12N 1/205; C12R 2001/01

(57) ABSTRACT

Isolated strains of Enterobacter kobei, Pantoea ananatis and Pantoea ananatis, comprising the characteristics of deposited strains CM-CNRG TB168, CM-CNRG TB169 and CM-CNRG TB171, respectively; wherein said bacterial strains are endophytes of Zea maysL. plants; and exhibit vegetable plant growth stimulant activity. Formulation for stimulating vegetable plant growth including at least one isolated bacterial strain, which is selected from the following group: CM-CNRG BT168, CM-CNRG BT169, CM-CNRG BT171, and mixtures thereof: at least one prebiotic substance: and at least one excipient. Method for stimulating the growth of vegetable plants including applying to vegetable plants, a sufficient amount of a formulation for stimulating the growth of vegetable plants, in accordance with the present invention.

### TECHNICAL FIELD OF THE INVENTION

The present invention relates to the technical field of Biotechnology and Agriculture, in that it provides endophytic bacterial strains of corn plant (Zea mays L.); probiotic mixtures comprising the endophytic strains; formulations and a method for stimulating the growth of vegetable plants, through the use of said formulation, which contains the bacterial strains.

### BACKGROUND OF THE INVENTION

The increase in the demand for agrochemical products, mainly fertilizer products and their excessive and indiscriminate use have not only increased the price of the product, but also cause soil erosion, decreasing the nutrients, health and fertility of the soil. Corn cultivation plays an important role worldwide, where Mexico is among the top 5 countries with the highest production. The growth of this crop is limited by the availability of nutrients found in the soil. This nutrient deficiency is reflected in the yield per hectare of the crop, in the susceptibility of the crop against pathogens, as well as contributing to environmental pollution. The use of microorganisms as biofertilizers has emerged as an alternative not only environmentally friendly, but also as an economical alternative to increase crop yields.

Mukherjee, A., et al. (Aug. 2020) identified cultivated endophytes from germinated and dry seeds of chickpea Cicer arietinum L., and their functional attributes. They isolated 29 bacterial strains from chickpea seeds (8 strains from dry seeds and 21 from germinated seeds). Phylogenetic analysis based on 16S rDNA showed that the endophytic seed bacteria belong to Enterobacter sp., Bacillus sp., Pseudomonas sp., Staphylococcus sp., Pantoea sp. and Mixta sp.

The isolates produced a significant amount of Indole-3-acetic acid (IAA) (Enterobacter hormaechei BHUJPCS-15; 58.91 μg/mL), solubilized phosphate (Bacillus subtilis BHUJPCS-24; 999.85 μg/mL) and potassium, ammonia (Bacillus subtilis BHUJPCS-BHUJPCS 12; 148.73 μg/mL), and also inhibited the growth of chickpea pathogen (Pseudomonas aeruginosa BHUJPCS-7 vs. Fusarium oxysporum f.sp. ciceris) under laboratory conditions. Several seed endophytes induced a significant increase in plant growth and increased tolerance of chickpea plants to the pathogen (Fusarium oxysporum f.sp. ciceris) when tested in vitro. Reintroduction of these isolates resulted in significant increases in plant length, biomass and chlorophyll content, and biocontrol activity against Fusarium oxysporum f.sp. ciceris. These results provide direct evidence for the presence of beneficial seed microbiomes and suggest that these isolates could develop into potential bioinoculants for improved disease management and sustainable increases in agricultural productivity. These strains were only tested in vitro, so it is very likely that these strains will not have the same effect when used in the field and in cereal crops.

Hernandez-Guisao, Rafael (Aug. 2019) in his thesis evaluated the plant growth promoting activity of Stevia rebaudiana endophytic bacteria on Medicago sativa and S. rebaudiana to select the best bacteria and establish conditions for their growth in Erlenmeyer flasks and bioreactor in a culture medium with industrial substrates, 12 strains previously isolated from S. rebaudiana were tested; which showed no improvement in the growth of S. rebaudiana plants, but Actinobacter sp. and 3 other bacteria increased the total content of steviol glycosides, while in M. sativa they improved plant growth by 60-120%. An analysis by principal component showed that the promoter characteristics of the bacteria are not related to their in planta activity in M. sativa and that the bacterium with the highest growth promotion was E. hormaechei, concluding that this last microorganism is a plant growth promoter with potential use in biofertilizers. These strains were also only tested in laboratory culture media, so there is no guarantee that they will have the same results when used in the field and in cereal crops.

Nowadays, it is common to isolate strains of microorganisms for different purposes. Due to the endemic characteristics of geographic regions, countries and/or territories, strategies are required to use microorganisms with specific and efficient activity and/or functions in those regions, countries and/or territories, where they are to be applied, as an advantage for the use of such isolated microorganisms. Therefore, in order to counteract the aforementioned drawbacks, endophytic bacterial strains were isolated from corn Zea mays L., which stimulate plant growth of plants, therefore, mixtures, formulations and methods were developed to stimulate plant growth of plants.

The characteristic details of the present invention are clearly shown in the following detailed description with the support of examples and figures which are annexed by way of illustration, for the purpose of illustrating the conception of said invention and some preferred embodiments, wherein:

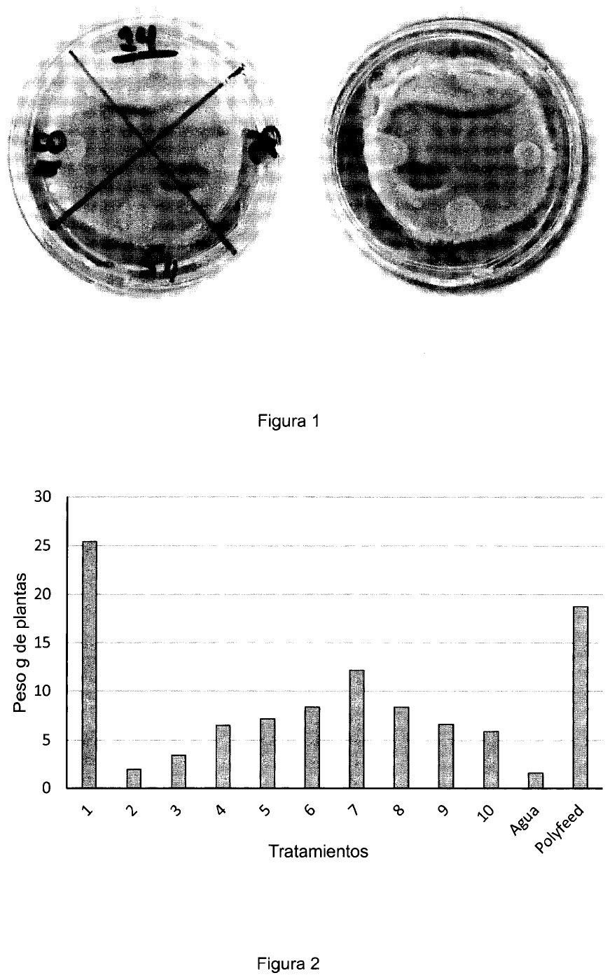

FIG. 1 is a photograph of the top and bottom view of a compatibility test between 3 selected bacterial strains, M14, M115 and M118. We can see that both M14, which is the bacterium below, and M115 and M118, which are above on the wafer, show good growth, indicating that the 3 strains are compatible with each other.

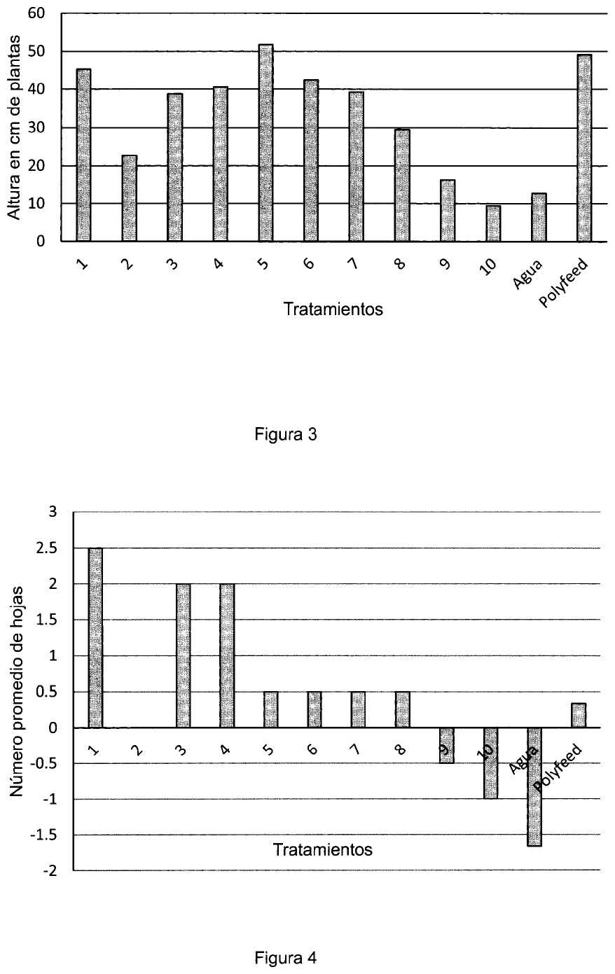

FIG. 2 is a graph showing the comparison of the average weights of the plants after inoculation with bacterial strains M14, M115, M118 and their mixtures.

FIG. 3 is a graph showing the comparative average plant height after inoculation with bacterial strains M14, M115, M118 and their mixtures.

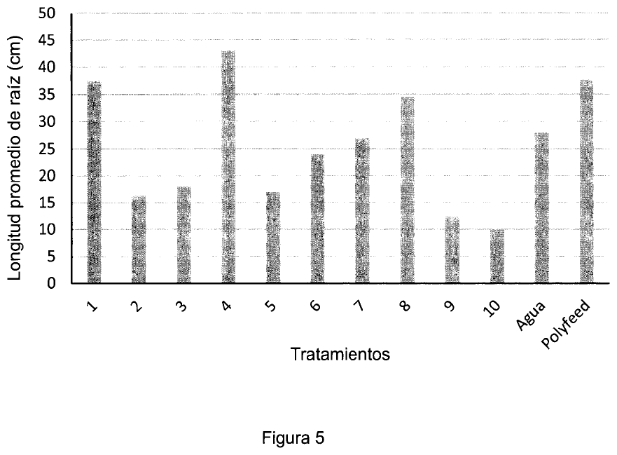

FIG. 4 is a graph showing the comparison of the average number of leaves of the plants after inoculation with bacterial strains M14, M115, M118 and their mixtures.

FIG. 5 is a graph showing the average root length comparison of plants after inoculation with bacterial strains M14, M115, M118 and their mixtures.

### DETAILED DESCRIPTION OF THE INVENTION

The present invention relates to a strain isolated from Enterobacter kobei, comprising the characteristics of the strain deposited under accession number CM-CNRG TB168.

A further object of the present invention is a strain isolated from Pantoea ananatis, which has the characteristics of the strain deposited under accession number CM-CNRG TB169.

Another item of this invention is a strain isolated from Pantoea ananatis, which has the characteristics of the strain deposited under accession number CM-CNRG TB171.

The bacterial strains isolated and deposited according to the present invention, CM-CNRG TB168 (Enterobacter kobei), CM-CNRG TB169 (Pantoea ananatis), and CM-CNRG TB171 (Pantoea ananatis), are endophytes of Zea mays L. plants; and it was found that these bacterial strains exhibit growth stimulating activity on vegetable plants, such as vegetable plants of the Poaceae family; specifically on vegetable plants of the Zea genus; more specifically on vegetable plants of the Zea mays L. species. The present invention also relates to a probiotic mixture, comprising at least two isolated bacterial strains, in accordance with the bacterial strains provided by the present invention.

A further object of the present invention is a formulation for stimulating growth in vegetable plants, wherein said formulation comprises:
i) at least one isolated bacterial strain, which is selected from the following group: CM-CNRG BT168, CM-CNRG BT169, CM-CNRG BT171 and their possible mixtures among them;
ii) at least one prebiotic substance; and
iii) at least one excipient.

One embodiment of the formulation of the present invention is where the bacterial strains, either individually or mixed together, are in an amount of 1%, the prebiotic substance in 1.5%, and the excipient in 97.5%, with respect to the total volume of the formulation.

Another object of the present invention is a method for stimulating the growth of vegetable plants, which comprises applying to vegetable plants a sufficient amount of a formulation for stimulating the growth of vegetable plants in accordance with the present invention.

One embodiment of the method for stimulating plant growth according to the present invention is when the sufficient amount of the formulation is 1 L per 80,000 vegetable plants.

A further variant of the method of the present invention is when the application of the formulation is in the vegetative stage of the vegetable plants.

A further embodiment of the method of the present invention is where the vegetable plants are of the family Poaceae, preferably of the genus Zea, and more preferably of the species Zea maysL.

### Examples

The following examples illustrate some embodiments of the present invention, therefore, they should not be considered as limiting the scope of protection of said invention.

### Example 1. Isolation of Endophytic Bacterial Strains from Corn (Zea mays L.) Plants

Whole plants of corn (Zea mays L.) with commercial maturity age V4 to R6, were collected from 2 plots of corn conventionally cultivated with agrochemical products, located in the towns of Santa Elena and San Francisco de Asis, municipality of Atotonilco el Alto, Jalisco, Mexico; and from a plot of corn cultivated with organic products, located in the town of El Refugio, municipality of Tototlan, Jalisco, Mexico.

A random sampling was carried out, taking 10 complete corn plants per plot, of healthy appearance, trying to damage the root as little as possible. This collection was carried out in different strategic points of the plots, in order to cover the total of their extensions to obtain representative samples.

The plants were transported in a plastic bag to avoid possible damage to the plants. Once they arrived at the laboratory, they were rinsed with running water and soap to remove excess soil and left to drain until the water was removed.

Once the plants were washed, they were sectioned by parts (root, stem and leaves) and seeds were taken from the cobs to later follow the endophyte extraction protocol, which changes depending on the section to be extracted.

For stem and leaf, they were first washed with 3% sodium hypochlorite for 10 min, followed by 3 washes with sterile double distilled water for 1 min each; while for root, they were first washed with 5% sodium hypochlorite for 10 min and finally, 3 washes with sterile double distilled water for 5 min each. For seeds, before washing with sodium hypochlorite, they were left to stand for two days in a 1% lime solution, and then followed the same procedure as for stem and leaf. 1 mL of water from the last washing was taken and placed in a petri dish of trypticasein soy agar to ensure that the washing was correct. Subsequently, the tissues were ground with the help of a mortar and isotonic saline solution, in a ratio of 1:2 for root, 1:2.5 for stem and 1:4 for leaf, until a suspension was formed, which was serially diluted to be plated on nutrient agar. For root, 6 dilutions were made and plated; and for seed, stem and leaf, 4 dilutions were made and plated.

As the suspension was diluted and plated, colonies formed less and less concentrated. From these dilutions, colonies with different morphologies were taken and reseeded 2 to 3 times in trypticasein soy agar until purified.

A total of 43 bacterial colonies with different characteristics and properties were isolated, which were identified as shown in Table 1, and were subjected to different agronomic tests.

### Example 2. Agronomic Tests to which the Endophytic Bacterial Strains of Corn Plants were Submitted

The 43 bacterial strains isolated from corn from the above example were subjected to the following agronomic tests: nitrogen fixation test (NFB), phosphate solubilization test (NBRIP Ca, NBRIP Al and NBRIP Fe), iron chelator test (siderophores), phytohormone production test (auxins) and acc deaminase enzyme (ACC) production test as described by Dworkin in 2006, Delvasto et al, in 2006, Milagres etal in 1999, Gordon and Weber in 1950, Penrose and Glick in 2003, respectively. In this case, as our main objective was to select microorganisms that induce plant growth, we emphasized the production of auxins, since this is a phytohormone related to growth and solubilization of calcium phosphates, as it is one of the key elements for the growth of this plant.

However, we also considered the results of the other tests to generate a product that not only achieves an adequate induction of plant growth, but also provides other properties that can be beneficial to the plant. The results of these agronomic tests are shown in Table 1.

When analyzing the results of the isolated bacterial strains (Table 1), we selected 3 bacterial strains, which were M14, M115 and M18, because they were positive for the nitrogen fixation test (NFB), due to the coloration change that was generated in the culture medium where they were sown. For the solubilization of phosphates, the halo generated around the inoculated colony in the medium was measured, the strain corresponding to M14 generated a halo of 0.1 cm in the solubilization of calcium phosphates, while strain M115 was able to solubilize calcium phosphates forming a halo of 0.2 cm, and finally strain M118 generated a halo of 0.3 cm, being the strain with the highest phosphate solubilization of the three selected strains. With aluminum and iron phosphates, none of the isolates, including the three selected strains, were able to solubilize these phosphates. In the case of siderophores, only strain M14 is positive for the production of these chelators.

In auxin production, the strain that produced the greatest amount of this phytohormone was strain M14, obtaining 3 out of 3 crosses, while strains M115 and M118 obtained a production of 2 out of 3 crosses, that is, they produced it moderately. Finally, the production of the enzyme acc deaminase, as shown in Table 1, strain M118 did not produce this enzyme because it was not able to grow in the culture medium, while strains M14 and M115 produced it in low quantities, obtaining only 1 cross out of 3.

TABLE 1
Results of agronomic tests of 43 endophytic bacterial
strains isolated from Zea mays L. plants.

| | | NBRIP | NBRIP | NBRIP | | | |
|---|---|---|---|---|---|---|---|
| Strain | NFB | Ca | Al | Fe | Siderophores | Auxins | ACC |
| M14 | + | 0.10 | − | − | + | +++ | + |
| M86 | + | − | − | − | − | − | + |
| M87 | + | lig+ | − | − | + | + | − |
| M88 | − | − | − | − | + | − | + |
| M89 | + | − | − | − | + | + | ++ |
| M90 | + | − | − | − | + | − | + |
| M91 | + | + | − | − | − | ++ | + |
| M92 | − | − | − | − | + | − | − |
| M93 | + | − | − | − | + | − | + |
| M94 | + | − | − | − | + | − | +++ |
| M95 | + | 0.10 | − | − | − | + | ++ |
| M96 | + | − | − | − | + | − | ++ |
| M97 | + | − | − | − | + | − | + |
| M98 | + | − | − | − | + | − | − |
| M99 | + | − | − | − | + | − | − |
| M100 | + | − | − | − | + | − | − |
| M101 | + | − | − | − | + | + | − |
| M102 | + | − | − | − | + | + | + |
| M103 | + | − | − | − | + | − | − |
| M104 | lig+ | − | − | − | + | − | − |
| M105 | + | − | − | − | − | + | + |
| M106 | + | − | − | − | + | − | + |
| M107 | lig+ | − | − | − | + | − | + |
| M108 | − | − | − | − | − | − | − |
| M109 | − | − | − | − | − | − | − |
| M110 | Not grown/contaminated | | | | | | |
| M111 | − | − | − | − | + | − | − |
| M112 | + | − | − | − | + | + | − |
| M113 | − | + | − | − | + | − | − |
| M114 | − | + | − | − | − | − | − |
| M115 | + | 0.20 | − | − | − | ++ | + |
| M116 | + | + | − | − | − | − | − |
| M117 | + | 0.10 | − | − | − | − | − |
| M118 | + | 0.30 | − | − | − | ++ | − |
| M119 | + | − | − | − | + | − | − |
| M120 | lig+ | − | − | − | + | − | − |
| M121 | + | − | − | − | + | − | − |
| M122 | + | lig+ | − | − | + | − | + |
| M123 | − | − | − | − | + | + | + |
| M124 | + | − | − | − | + | − | + |
| M125 | + | − | − | − | + | − | − |
| M126 | + | − | − | − | + | − | + |
| M127 | lig+ | − | − | − | + | − | − |

Where: ig + is minimum production, + is low production, ++ is moderate production, +++ is abundant production, and NC = No Growth.

Therefore, according to the results obtained from bacterial strains 5 (Table 1), only bacterial strains M14, M115 and M18 were selected because they produced auxins, were positive for N fixation, were able to solubilize Ca phosphates, were positive for the production of siderophores (M14) and were able to produce the enzyme ACC deaminase, although in small quantities in strains M14 and M115. Therefore, we proceeded to identify 10 of these 3 selected bacterial strains. Therefore, we proceeded to the identification of these 3 selected bacterial strains.

### Example 3. Identification of the 3 Endophytic Bacterial Strains of Zea may L. Selected

Once the 3 bacterial strains of the previous example were selected, we proceeded to identify them. The bacterial identification was carried out by MALDI-TOF by MALDI-TOF technique, which consists of obtaining a protein profile of the microorganism analyzed and to make a comparison with a database resulting in a score, which corresponds to the accuracy of the identification. The score ranges from 0 to 3, where a score of 01.599 indicates that it is not a reliable identification, a score of 1.6-1.999 indicates that there is a high probability that the identified genus is the correct one, a score of 2.02.299 indicates that the identified genus is 100% correct, while the species has a certain probability, and finally, a score of 2.300-3.000 indicates that the identified genus and species are 100% correct. The identification results obtained are shown in Table 2.

TABLE 2
Identification of the 3 strains of endophytic
bacteria from Zea may L.

| Cepa | Identification | Score |
|---|---|---|
| M14 | Enterobacter kobei | 1.970 |
| M115 | Pantoea annanatis | 2.150 |
| M118 | Pantoea annanatis | 2.210 |

### Example 4. Compatibility Test Between Selected Endophytic Bacterial Strains of Zea may L. Plants

Once the 3 bacterial strains selected in the previous example were identified, we proceeded to perform the compatibility test between them, to see the feasibility of being able to make mixtures and measure the effect on germination.

Compatibility tests were performed using the sandwich technique, where a bacterium is sown as a lawn in a box of casoy agar and on top of the inoculated bacterium, a casoy agar wafer is placed, where the rest of the bacteria to be tested are inoculated in the form of spots. If the bacteria on top of the wafer grows, it is considered to be compatible with the bacteria underneath the wafer. the bacteria underneath the wafer. All this is done in triplicate.

The results of this compatibility test can be seen in FIG. 1, which indicated that the 3 selected bacterial strains (M14, M115 and M118) showed good growth with each other, proving that the 3 strains (M14, M115 and M118) are compatible and that they can be used in mixture to achieve their effect of stimulating germination in vegetable seeds.

### Example 5. Ex Vitro Growth Induction Tests of the Endophytic Bacterial Strains of Selected Zea may L. Plantss

For the ex vitro growth induction tests we used the 3 selected strains: M14, M115 and M118 and with the help of a statistical software we made a design of experiments that indicated all the possible mixtures. This resulted in a total of 10 treatments with all the probable mixtures involving the 3 strains. In addition, 2 controls, field nutrition (polyfeed) and water, were used. The treatments tested are described in Table 3.

Two corn (Zea mays L.) seeds were used for each treatment, and they were germinated in a germination tray in the dark with constant irrigation until the first seedlings emerged and reached an average height of 5 cm. an average height of 5 cm. Subsequently, these already grown seedlings were transferred to pots with a substrate composed of soil, peatmoos and jal and were kept outdoors and watered every 2 days. Before starting the inoculation, the plants were taken out of the pots making sure not to damage the roots, then they were washed with running water to remove excess soil and were weighed, measured and the number of initial leaves was counted; then 2 plants per treatment with similar weights were taken, numbered and transplanted back into their respective pots.

TABLE 3
Treatments to which corn (Zea may L) seeds were
subjected with the isolated bacterial strains.

| Treatments | Description |
|---|---|
| T1 | M14 |
| T2 | M14 + M115, proportion 2:1 |
| T3 | M14 + M118, proportion 2:1 |
| T4 | M14 + M115, proportion 1:2 |
| T5 | M14 + M115 + M118, proportion 1:1:1 |
| T6 | M14 + M118, proportion 1:2 |
| T7 | M115 |
| T8 | M115 + M118, proportion 2:1 |
| T9 | M115 + M118, proportion 1:2 |
| T10 | M118 |
| T11 | Control (water only) |
| T12 | Polyfeed |

The bacteria to be tested were inoculated individually in a conventional culture medium containing carbon and nitrogen sources designed for their production, and incubated for 24 to 48 h, then, with the aid of sterile falcon tubes, the 10 mixtures were made. With a 5 mL micropipette and sterile tips, 5 mL of bacterial suspension were inoculated per plant. Two inoculations were carried out during a period of 1 month. After the inoculation period, the plants were taken out of their pots, washed with running water and weighed, measured, the root was measured and the final number of leaves was counted; these results are presented and discussed below.

Table 4 shows the concentration of the measured marameters, where the data reported are the set of data measured before and after inoculation, except for the root. The root data are only post inoculation.

TABLE 4
Parameters measured in corn plants.

| Treatment | Weight (g) | Leaves | Height (cm) | Root (cm) |
|---|---|---|---|---|
| 1 | 25.442 | 2.5 | 45.25 | 37.5 |
| 2 | 1.942 | 0 | 22.75 | 16.25 |
| 3 | 3.442 | 2 | 38.75 | 18 |
| 4 | 6.542 | 2 | 40.5 | 43 |
| 5 | 7.192 | 0.5 | 51.75 | 17 |
| 6 | 8.442 | 0.5 | 42.5 | 24 |
| 7 | 12.192 | 0.5 | 39.25 | 27 |
| 8 | 8.442 | 0.5 | 29.5 | 34.5 |
| 9 | 6.642 | −0.5 | 16.25 | 12.5 |
| 10 | 5.942 | −1 | 9.5 | 10 |
| Polyfeed | 18.77533333 | 0.333333333 | 49.16666667 | 37.6666667 |
| Water | 1.608666667 | −1.666666667 | 12.83333333 | 28 |

When observing the weights of the plants after being inoculated with the different bacterial mixtures, the best treatment was No. 1, which had an average weight of 25.442 g, followed by treatments 7, 6 and 8 with an average weight increase of 12.192, 8.442 and 8.442 g, respectively. On the other hand, the polyfeed and water controls had an average weight of 18.775 and 1.609 g, respectively, both are below the average weight of treatment 1; but only the water control is below all the treatments tested, in FIG. 2 the behavior of this parameter is better observed.

In the case of average plant height, the treatment that induced the best plant growth was treatment 5, with an average of 51.75 cm, followed by treatments 1 and 6, with an average height of 45.25 and 42.5 cm, respectively. The polyfeed and water controls reported an average height of 49.16 and 12.83 cm, respectively. The treatment with nutrition is below treatment 5 but above the rest of the treatments, while water is below all treatments except treatment 10, as shown in FIG. 3. The worst treatment for inducing plant growth is treatment 10, which is below the average height of the water control, with an average height increase of 9.5 cm.

FIG. 4 shows the comparative of the average number of leaves of the different treatments and the controls, where we observe that the treatment with the highest average number of leaves was treatment 1 with a total of 2.5 new leaves, followed by treatments 3 and 4 with an average of 2 leaves each.

In the case of the controls, polyfeed and water, they had a total of 0.3 and -1.61 leaves respectively, this is indicative that these treatments did not induce the formation of new leaves in the plants, since at least 7 of the 10 treatments are better in the induction of leaf production. The worst treatments are treatment 9 and 10, which, like the water treatment, do not induce the production of leaves, but dry the leaves that the plant had previously.

FIG. 5 clearly shows the results of the average root length, where we observe that the treatment that induced the highest root generation was treatment 4, with a root length of 43 cm, followed by treatments 1 and 8, which had a root length of 37.5 and 34.5 cm, respectively. Polyfeed achieved a root length of 37.66 cm, while the water control generated a root length of 28 cm, both below the best treatment, number 4. The treatments below the water control are 2, 3, 5, 6, 7, 9 and 10. All the above data and the other parameters are detailed in Table 4.

### Storage of Isolated Bacterial Strains

Seeing that the isolated bacterial strains M14, M115 and M118, have agronomic potential, we proceeded to deposit them on Oct. 19, 2020, in the Collection of Microorganisms of the National Center of Genetic Resources that belongs to the National Institute of Forestry, Agricultural and Livestock Research, with registration number before the World Federation of Culture Collection 1006 (CM-CNRG) and International Depositary Authority with notification 308 for purposes of patent procedure under the Budapest Treaty; and is domiciled at Boulevard de la Biodiversidad 400, C. P. 7600. Tepatitlan de Morelos, Jalisco, Mexico.

Bacterial strain M14 Enterobacter kobei was assigned deposit number CM-CNRG BT168, strain M115 Pantoea ananatis was assigned accession No. CM-CNRG BT169, and strain M118 Pantoea ananatis was assigned accession No. CM-CNRG BT171.

Literature Cited Dworkin, M.; Falkow, S.; Rosemberg, E.; Schleifer, K.H. y Stackebrandt, E. (2006). Non-Symbiotic nitrogen fixers. The prokaryotes Vol. 5: Proteobacteria: Alpha and Beta subclasses, 3a ed., 69-89.

Delvasto, P., Valverde, A., Ballester, A., Igual, J. M., Munoz, J. A., Gonzalez, F., Blazquez, M.L. y Garcia, C. (2006). Characterization of brushite as a re-crystallization product formed during bacterial solubilization of hydroxyapatite in batch cultures. Soil Biology and Biochemistry, 38, 2645-2654. doi: 10.1016/j.soilbio.2006.03.020.

Milagres, A. M. F., Machuca, A. y Napoleao, D. (1999). Detection of siderophore production from several fungi and bacteria by a modification of chrome azurol S (CAS) agar plate assay. Journal of Microbiological methods, 37(1), 1-6.

Gordon, S. A., y Weber, R. P. (1950). Colorimetric estimation of indoleacetic acid. Plant Physiology, 192-195.

Penrose, D. M., & Glick, B. R. (2003). Methods for isolating and characterizing ACC deaminase-containing plant growth-promoting rhizobacteria.

Physiol Plant, 118(1), 10-15.

1. A composition comprising one or more bacterial isolate strains, the one or more bacterial isolate strains comprising Enterobacter kobei isolate; comprising a strain deposited under accession number CM-CNRGTB168, a strain isolated from Pantoea ananatis comprising the strain deposited under accession number CM-CNRG TB169. A strain isolated from Pantoea ananatis comprising the strain deposited under accession number CM-CNRG TB171, or any combination thereof.

2. (canceled)

3. (canceled)

4. The composition of claim 1, wherein said bacterial isolate strains are endophytes of plants of Zea mays L.

5. The composition of claim 1, wherein the bacterial isolate strains exhibit plant growth stimulating activity in plant growth.

6. The composition of claim 5, wherein the plant is a vegetable plants are of the family Poaceae.

7. The composition of claim 6, wherein the vegetable plants are of the genera Zea.

8. The composition of claim 7, wherein the vegetable plants are of the species Zea mays L.

9. A probiotic mixture, comprising at least two isolated bacterial isolate strains of claim 1.

10. The probiotic mixture of claim 9, wherein said bacterial isolate strains are endophytes of plants of Zea mays L.

11. The probiotic mixture of claim 9, wherein such bacterial strains exhibit stimulating activity on the growth of vegetable plants.

12. The probiotic mixture of claim 11, wherein the vegetable plants are of the family Poaceae.

13. The probiotic mixture of claim 12, wherein the vegetable plants are of the genus Zea.

14. The probiotic mixture of claim 13, wherein the vegetable plants are of the species Zea mays L.

15. A formulation for stimulating growth in vegetable plants, comprising:
i) at least one isolated bacterial strain, which is selected from the following group: CM-CNRG BT168, CM-CNRG BT169, CM-CNRG BT171, and their mixtures with each other;
ii) at least one prebiotic substance; and
iii) at least one excipient.

16. The formulation of claim 15, wherein the bacterial strain, either individually or mixed, is in an amount of 1%, the prebiotic substance in 1.5%, and the excipient in 97.5%, with respect to the total volume of the formulation.

17. The formulation of claim 15, wherein the vegetable plants are from the family Poaceae.

18. The formulation according to claim 17, wherein the vegetable plants are of the genus Zea.

19. The formulation of claim 18, wherein the vegetable plants are of the species Zea mays L.

20. A method for stimulating the growth of vegetable plants, comprising, applying to vegetable plants, a sufficient amount of a formulation for stimulating the growth of vegetable plants, according to claim 15.

21. The method of claim 20, wherein the sufficient amount of the formulation is 1 L per 80,000 vegetable plants.

22. The method according to claim 20, wherein the application of the formulation is in the vegetative stage of vegetable plants.

23. The method of claim 20, wherein the vegetable plants are of the family Poaceae.

24. The method according to claim 23, wherein the plants are of the genus Zea.

25. The method of claim 24, wherein the vegetable plants are of the species Zea mays L.
